(19) United States
(12) Patent Application Publication
(10) Pub. No.: US 2025/0308071 A1
(43) Pub. Date: Oct. 2, 2025

(54) METHOD FOR DETERMINING THE SPORE COLOR INDEX (SCI) IN ORGANOPALYNOLOGICAL SLIDES

(71) Applicants: PETRÓLEO BRASILEIRO S.A. - PETROBRAS, Rio de Janeiro (BR); PONTIFÍCIA UNIVERSIDADE CATÓLICA DO RIO DE JANEIRO, Rio de Janeiro (BR)

(72) Inventors: ANDRE LUIZ DURANTE SPIGOLON, Rio de Janeiro (BR); KAREN SOARES AUGUSTO, Rio de Janeiro (BR); ITALO DE OLIVEIRA MATIAS, Rio de Janeiro (BR); LEANDRA COSTA LAGES, Rio de Janeiro (BR); RICHARD BRYAN MAGALHÃES SANTOS, Rio de Janeiro (BR); MARCOS HENRIQUE DE PINHO MAURICIO, Rio de Janeiro (BR); IGOR VIEGAS ALVES FERNANDES DE SOUZA, Rio de Janeiro (BR); GIL MARCIO AVELINO SILVA, Rio de Janeiro (BR); SIDNEI PACIORNIK, Rio de Janeiro (BR)

(73) Assignees: PETRÓLEO BRASILEIRO S.A. - PETROBRAS, Rio de Janeiro (BR); PONTIFÍCIA UNIVERSIDADE CATÓLICA DO RIO DE JANEIRO, Rio de Janeiro (BR)

(21) Appl. No.: 19/091,057

(22) Filed: Mar. 26, 2025

(30) Foreign Application Priority Data
Mar. 27, 2024 (BR) 1020240061420

(51) Int. Cl. G06T 7/90; G01N 21/25; G01N 33/24; G06V 10/70; G06V 20/69

(52) U.S. Cl. CPC G06T 7/90; G01N 21/251; G01N 33/243; G06V 10/70; G06V 20/698; G06T 2207/10024; G06T 2207/10056; G06T 2207/20092

(57) ABSTRACT

The invention comprises a method for determining the spore color index (SCI) in organopalynological slides. The slide is loaded into a motorized optical microscope that automatically scans and takes images. A previously trained Deep Learning-based artificial intelligence automatically identifies sporomorphs in the images obtained and a computer calculates the SCI of each image based on a calibration equation obtained with standard SCI slides. This equation correlates the intensity of the red color channel of each sporomorph and its respective SCI, allowing an automatic calculation for other sporomorphs captured under the same lighting conditions.

### CROSS-REFERENCE FOR RELATED APPLICATIONS

This application claims priority to Brazilian Application No. BR 1020240061420, filed on Mar. 27, 2024, the disclosure of which is herein incorporated by reference in the entirety.

### FIELD OF THE INVENTION

The present invention is part of the field of petroleum engineering. More specifically, the present invention is related to methods for automatic identification by an artificial intelligence (AI) of sporomorphs in a rock sample and automatic measurement of the spore color index (SCI) in organopalynological slides.

### BACKGROUND OF THE INVENTION

Determining the degree of maturation and thermal evolution in kerogen is of vital importance to the oil industry, as it allows estimating whether the sample from a given depth in an oil well is in the oil or natural gas generation window or whether it is still immature. There are several ways to assess the degree of thermal maturation. One of them is through the spore color index (SCI) in organopalynological slides.

It is known that spore color is closely linked to thermal maturation because, as the physical-chemical processes advance to a field of greater maturation, the spores show a color change with lower transmittance. In other words, the darker the palynomorphs, the greater their maturation. The difficulty in estimating this color arises from the subjectivity and time required for such analysis. The analysis depends on an operator trained to identify the spores, who must manually investigate the organopalynological slides in search of sporomorphs (spores and pollen grains), and visually, a value must be assigned to each one based on a visual pattern.

Currently, SCI analysis is performed manually by a specialized operator. The operator uses an optical microscope in transmitted light mode, investigating organopalynological slides and identifying possible sporomorphs. Once identified, the operator uses standard slides in the presumed SCI range to visually assess the best value for the sporomorph under analysis. The standard consists of a set of slides, each containing a sporomorph with a specific SCI value, each slide being labeled with a scale between 1.0 and 10.0, with a gradation of 0.5. The higher the value, the greater the degree of thermal maturation of the sporomorph.

At the end of the analysis, a histogram is generated with the SCI measurements of all identified sporomorphs. Then, a subset of these sporomorphs is selected that the operator identifies as being representative of the sample (they are not contaminated, not degraded, not reworked). These spores resulting from the interpretation generate a new histogram and its mode represents the SCI of the sample.

This process is entirely manual and time-consuming, in addition to being subject to the subjectivity of the human operator.

### STATE OF THE ART

The document U.S. Pat. No. 5,233,409, entitled “Color analysis of organic constituents in sedimentary rocks for thermal maturations”, discloses that the thermal maturity state of organic matter extracted from a sedimentary rock sample is determined using a microscope in combination with a color video camera to provide an image of the organic matter in transmitted and filtered light, using the camera output to calculate RGB and HSB values for a selected area of the image per pixel and plotting integrated values over the selected area in the form of a thermal maturation path from which the probability that petroleum has been generated in the rock can be determined.

More specifically, this document discloses methods for determining the color of organic matter extracted from a rock sample, which include the steps of using a high-power microscope and a high-resolution color video camera to photograph a slide-mounted specimen under transmitted illumination and connecting the camera output to a frame grabber board and processor in a digital computer programmed to acquire values representing the above-mentioned red, green, blue (RGB) and hue, saturation, and brightness (HSB) parameters. After the system has been calibrated with a known color value standard, an image of the specimen is captured on a multi-synchronized remote monitor where the area to be examined, which may be a spore, for example, is outlined by a remote operator. In this way, the RGB components of the color are measured. Mechanical computation is used to calculate the corrected HSB values, which are converted to maximum temperature values and expressed as a thermal maturity level so as to reflect the degree of thermal evolution of the sedimentary organic matter (kerogen). The results are generally represented in histogram form and can be made available to illustrate a single object, an entire sample and/or an entire well log.

The document EP 3596638 A1, entitled “Collaborative sensing and prediction of source rock properties”, discloses computer-implemented systems, apparatus and methods for the detection and prediction of source rock properties. A method for predicting the maturity of a source rock sample is disclosed, which includes the application of a set of analytical techniques and obtaining various data. A predictive correlation is generated by applying a machine learning model to the acquired data. Subsequently, the collected data is processed using this correlation, thus allowing the determination of the maturity of the source rock sample.

This document also discloses that spectroscopy measurements involve a light source, the reflection of light or the transmission of light through a sample and the detection of the light intensity by a detector. In addition, the source is either monochromatic or the detector is wavelength selective, so that the attenuation of light (whether by reflection or transmission) can be observed as a function of wavelength. Current laboratory spectroscopy instruments for mid- and long-infrared (IR) wavelengths typically use semiconductor photodiode detectors. These detectors are made of materials such as mercury cadmium telluride, indium gallium arsenide, or indium arsenide, which must be cooled below room temperature (e.g., thermoelectrically or with liquid nitrogen) to obtain usable signal-to-noise ratios.

### SUMMARY OF THE INVENTION

The objective of the present innovation is to automate most or all of the analysis of the spore color index (SCI) measurement to contribute to the assessment of the degree of thermal maturation of the sample, saving time and efficiency. A system based on “Deep Learning” was trained for the automatic identification of sporomorphs (spores and pollen grains). With the help of an expert operator in the area, several images were captured, and the spores and pollen grains were manually outlined to form a database with images of different wells, depths and SCI values. This database was separated into a training set and a validation set, allowing the network to learn and then have its performance evaluated. The method described herein obtained approximately 90% accuracy in validation tests.

### BRIEF DESCRIPTION OF THE DRAWINGS

FIG. 1 is a representation of a graph correlating the intensity of the red channel and the SCI value of samples of the SCI standard, according to the present invention. It is worth noting that the images were captured in 8-bit RGB, which generates 256 possible tones for the red channel.

FIG. 2 is a representation of an automated optical microscope.

FIG. 3 is a flowchart of the method according to the present invention.

### DETAILED DESCRIPTION OF THE INVENTION

Specific embodiments of the present disclosure are described below. In an effort to provide a concise description of these embodiments, all features of an actual implementation may not be described in the specification. It should be appreciated that in the development of any actual implementation, as in any engineering or design project, numerous implementation-specific decisions must be made to achieve the specific objectives of the developers, such as compliance with system-related and business constraints, which may vary from one implementation to another. Furthermore, it should be appreciated that such a development effort may be complex and time-consuming but would nevertheless be a routine design and manufacturing undertaking for those of ordinary skill having the benefit of this disclosure.

The first aspect of the present invention comprises a method of identifying sporomorphs obtained from kerogen concentrate slides (i.e., organopalynological slides), while the second aspect comprises the calculation of the SCI. As previously mentioned, the sample analysis steps have heretofore been performed entirely manually by a human practitioner. This prior art solution is time-consuming, and its outcome is influenced by the subjectivity of the practitioner.

To overcome this difficulty, a system based on “Deep Learning” was trained for the automatic identification of sporomorphs. With the help of an operator specialized in the area, several images of sporomorphs were captured and the spores and pollen grains were manually outlined to form a database with images of different wells, depths and SCI values. This database was separated into a training set and a validation set, allowing the artificial intelligence (AI) to learn and then have its performance evaluated (with accuracy rates of approximately 90%). The image capture conditions were standardized so as to be always reproducible.

Any type of neural network could have been used for the aforementioned learning, for example, Mask R-CNN, which is a convolutional neural network, as well as networks such as U-Net and its variations (U-Net++, for example). However, the present invention is not limited to any particular type of neural network or class of algorithm, as long as it is a model that meets the Deep Learning specifications. However, it is worth noting that, for the present invention, Mask R-CNN was the algorithm that showed the best results.

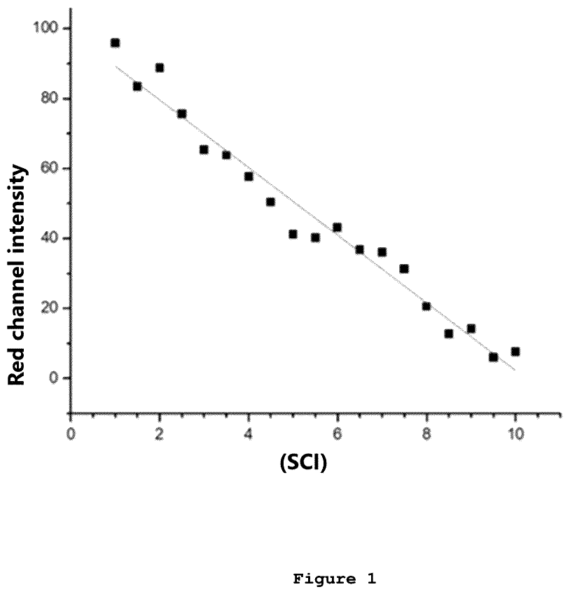

To avoid the frequent need to use the standard slides, images of them were captured in order to analyze the average intensity values of the red channel of each sporomorph. Several tests corroborated the indication in the literature that the red channel is the one that best correlates with thermal maturation. Each standard slide was analyzed under an optical microscope to determine the intensity of the red channel. With this, the graph in FIG. 1 was created and the line representing the median of the points was calculated according to equation (1):

y=a+b*x (1)
wherein “y” is the intensity of the red channel, “x” is the SCI, “a” is a constant with a value of 99.02±2.52, and “b” is a constant with a value of −9.67±0.41. Equation (1) is the calibration equation for the optical microscope used.

Thus, a linear correlation was calculated using the least squares method between the mean intensity of the red channel and the SCI (both from the standard slides), obtaining a coefficient of determination (R2) of 0.97 for the predefined operating conditions of the optical microscope. This coefficient is an assessment of how close the proposed linear correlation is to the real points, with a coefficient of 1.0 being a perfect correlation. Thus, an R2 coefficient of 0.97 means an excellent correlation between the equation and the data obtained from the standard slides. With the coefficients a and b, the SCI can be calculated directly from the mean intensity of the red channel of the sample of sporomorphs previously outlined and identified by the trained AI mentioned above.

This allows the SCI to be calculated automatically for the first time without the need for visual inspection by a human. However, any change in microscope conditions requires a new adjustment of this linear correlation. The angular coefficient b is a property of the color of the sporomorphs and should not vary significantly, but the linear coefficient a depends on the lighting conditions used during capture, which may vary depending on the operator. Therefore, calibration is always necessary for SCI calculation.

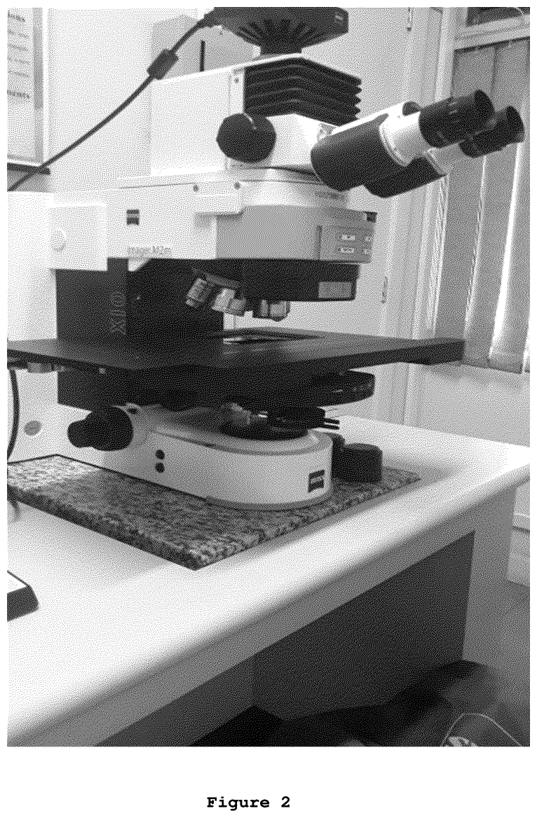

Preferably, an automated optical microscope capable of automatically scanning organopalynological slides is used, exemplified by the automated optical microscope with transmitted light and motorized XY stage in FIG. 2. The use of automated microscopes allows automatic capture of images under acquisition conditions similar to those used for the SCI calculation n (minimizing the need for daily recalibrations). This further speeds up the procedure and gives greater reliability to image acquisition. For the purposes of this disclosure, it is assumed that an automated optical microscope is used, but the present method is not limited by this. Conventional optical microscopes can be used, and the image capture can be performed manually.

The images captured by the optical microscope are fed to a computer configured to run the previously trained AI model. This model identifies the sporomorphs and the computer determines the average intensity of the red channel of the identified sporomorph. Based on equation (1), the computer calculates the SCI of each image analyzed. At the end of the analysis of all images, the SCI values obtained are organized into a distribution histogram for the sample analyzed.

Optionally, data refinement can be performed. The images taken from each sample can be made available so that an operator can interpret which is the most representative subset of these images (those whose sporomorphs are not contaminants, are not degraded and have not been reworked). Thus, the SCI is recalculated only for this subset of images, so that an additional SCI histogram of the most representative sporomorphs is also generated.

Finally, based on the histogram selected from the complete histogram or the histogram based only on the most significant subset of images, the SCI value of the slide is obtained using the mode.

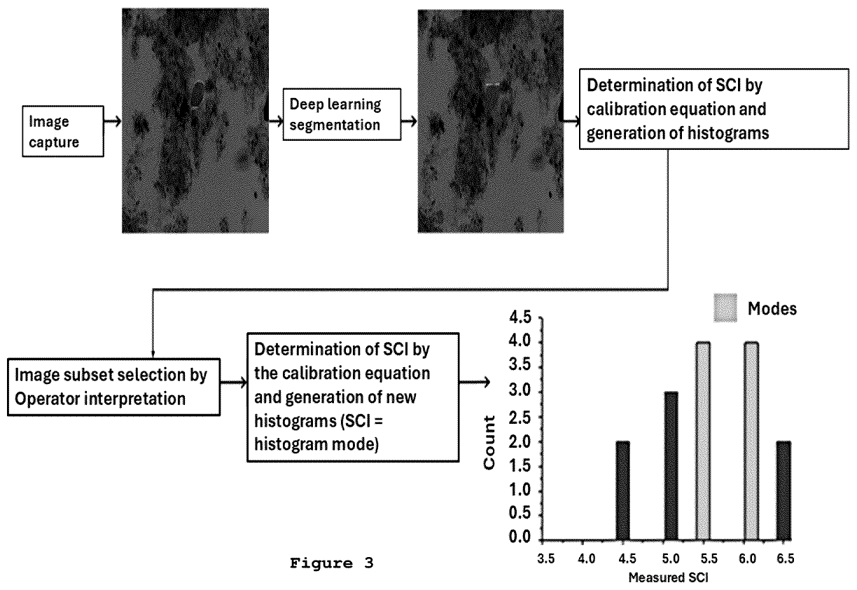

In summary, the method according to the present invention comprises the steps of, with reference to FIG. 3:
S1. Loading an organopalynological slide into a transmitted light optical microscope. Preferably, the optical microscope is fully motorized.
S2. Activating the scanning and image capture routine dedicated to organopalynological slides, capturing images at each visual field of analysis of the microscope.
S3. The images are saved in a memory for later evaluation.
S4. Identification of sporomorphs (spores and pollen grains) in each image by the trained AI model executed by a previously configured computer.
S5. Calculation, by the computer, of the SCI of each sporomorph of each image based on equation (1).

The above steps promote a major advance in the determination of the SCI of sporomorph samples. Based on the innovative steps above, the determination of the SCI of an organopalynological sample can be done much faster and more reliably, contributing significantly to the assessment of the degree of maturation of a sample.

After the above steps, the determination of the final SCI of the sample can be done in a manner equal or similar to the state of the art:
S6. Generation, by the computer, of a histogram of the distribution of the SCI values calculated for each sporomorph identified in the analyzed slide.
S7. Optionally, checking and selection by a human operator of a subset of the most significant sporomorphs in the images captured from a slide.
S8. Optionally, generation of a new histogram of SCI distribution based on the subset of the most significant sporomorphs.
S9. Indication of the Final/interpreted SCI of the slide from the histogram mode.

The advantages provided by the present invention are evident to the person skilled in the art.

The greatest advantage of the present invention is to automate and speed u analysis. Consequently, automated capture allows for faster productivity gains and aids in the development of other tasks.

Automatic identification and histogram generation leave the operator with only the optional task of determining, in the captured images, which sporomorphs would be the most representative for the SCI measurement of that sample. Not only is the time savings significant, but automatic scanning by an automated optical microscope also allows for a complete and more efficient analysis of the organopalynological slide.

Furthermore, the innovation described herein allows for the standard SCI slides not to have to be used in each analysis, preserving them. It is known that this standard is difficult to obtain, so the fact that the slides do not need to be used at all times protects them from possible damage during handling.

Although aspects of the present disclosure may be susceptible to various modifications and alternative forms, specific embodiments have been shown by way of example in the drawings and have been described in detail in this document. However, it should be understood that the invention is not intended to be limited to the particular forms disclosed. Rather, the invention must cover all modifications, equivalents and alternatives that fall within the scope of the invention as defined by the following appended claims.

1. A method for automatically or semi-automatically determining the spore color index (SCI) in organopalynological slides, comprising the steps of:
a) identification, by a trained artificial intelligence (AI) executed by a computer, of sporomorphs in images obtained from an organopalynological slide by a transmitted light optical microscope; and
b) calculation, by the computer, of the SCI of each sporomorph of each image based on a calibration equation that correlates the SCI with the intensity of the red channel of the sporomorph according to equation (1):
y=a+b*x (1)
wherein y is the intensity of the red channel, x is the SCI, a is a first constant with a value of 99.02±2.52, and b is a second constant with a value of −9.67±0.41.

2. A method, according to claim 1, further comprising the steps of:
c) generation, by the computer, of a histogram of distribution of the SCI values calculated for each sporomorph identified on the slide;
d) checking and selection, by a human operator, of a subset of the most significant sporomorphs in the images captured from a slide;
e) repeating step (c) based on the subset of the most significant sporomorphs; and
f) indicating a final SCI of the slide from the mode of the histogram obtained in the previous step.

3. The method of claim 1, wherein the optical microscope is an automated optical microscope and the method further comprises, before step (a), the steps of:
loading the organopalynological slide into the optical microscope;
activating a scanning and image capture routine dedicated to organopalynological slides; and
save the images obtained in the previous step in a memory.
